(19) United States
(12) Patent Application Publication
(10) Pub. No.: US 2023/0416209 A1
(43) Pub. Date: Dec. 28, 2023

(54) COMPOUNDS AND METHODS TO TARGET GLUCOSE-STIMULATED PHOSPHOHISTIDINE SIGNALING AND ESOPHAGEAL CANCER GROWTH

(71) Applicant: Health Research, Inc., Buffalo, NY (US)

(72) Inventors: Steven HOCHWALD, East Aurora, NY (US); Jianliang ZHANG, Williamsville, NY (US)

(21) Appl. No.: 18/253,384

(22) PCT Filed: Nov. 23, 2021
(86) PCT No.: PCT/US2021/060558

§ 371 Date: May 17, 2023

Related U.S. Application Data
(60) Provisional application No. 63/117,432, filed on Nov. 23, 2020.

(51) Int. Cl. C07D 253/07; C07D 487/04; C07C 233/81; C07D 307/92; C07D 271/113; C07D 239/74; C07D 209/42; C07D 307/93; C07D 239/62; C07D 493/04; C07D 239/70; C07C 49/643; C07D 257/02; C07D 311/20; C07D 471/06; C07C 281/08; C07D 327/08; A61K 33/243; A61P 35/00; A61K 31/53; A61K 31/196; A61K 31/365; A61K 31/4245; A61K 31/517; A61K 31/404; A61K 31/515; A61K 31/37; A61K 31/122; A61K 31/55; A61K 31/4748; A61K 31/175; A61K 31/382

(52) U.S. Cl. CPC C07D 253/07; C07D 487/04; C07C 233/81; C07D 307/92; C07D 271/113; C07D 239/74; C07D 209/42; C07D 307/93; C07D 239/62; C07D 493/04; C07D 239/70; C07C 49/643; C07D 257/02; C07D 311/20; C07D 471/06; C07C 281/08; C07D 327/08; A61K 33/243; A61P 35/00; A61K 31/53; A61K 31/196; A61K 31/365; A61K 31/4245; A61K 31/517; A61K 31/404; A61K 31/515; A61K 31/37; A61K 31/122; A61K 31/55; A61K 31/4748; A61K 31/175; A61K 31/382; C07C 2603/40; C07C 2603/86

(57) ABSTRACT

Provided are compounds and compositions that inhibit glucose-induced growth signaling and methods of using same. The compounds may be suitable to treat glycolytic cancers, such as, for example, esophageal squamous cell carcinoma (ESCC). The compounds may be used to inhibit or partially inhibit glucose-promoted tumor cell proliferation, NME-1 catalyzed histidine phosphorylation of FAK, and FAK interaction with RBI. The compounds may have the following structure:

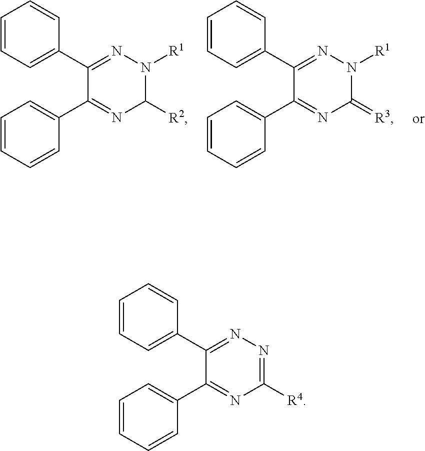

### CROSS REFERENCE TO RELATED APPLICATIONS

This application claims priority to U.S. Provisional Application No. 63/117,432, filed on Nov. 23, 2020, the disclosure of which is incorporated herein.

### BACKGROUND OF THE DISCLOSURE

Esophageal squamous cell carcinoma (ESCC) tumors are genetically heterogeneous, but these tumors nevertheless share a common metabolic weakness, i.e., albeit growth factor-independent, their proliferation is glucose (Glc)-dependent. The Glc levels required to induce ESCC proliferation is ˜160-fold lower than that found in normal blood, which further indicates that Glc functions in ESCC as a growth factor-like mitogen. Previous attempts by others to block Glc uptake by targeting receptor tyrosine kinases, which directly impacts Glc uptake/metabolism, have had limited success, due to significant side-effects.

### SUMMARY OF THE DISCLOSURE

The present disclosure describes targeting Glc-induced growth signaling, but not Glc uptake metabolism, to prevent ESCC growth without possessing major toxicity concerns. Targeting this pHis pathway may be used to prevent Glc-induced ESCC progression associated with the lack of ESCC response to GFI therapies by blocking the FAK-RB1 interaction (FIG. 1). The technology therefore imparts active lead compounds that inhibit Glc-stimulated pHis58-FAK through the inhibition of NME1-catalyzed histidine phosphorylation, while also interrupting the Glc-induced FAK-RB1 interaction.

Targeting Glc-induced growth signaling, which is prevalent in ESCC—but not Glc uptake/metabolism—which is prevalent in normal cells, the present disclosure aims to prevent ESCC growth, without the underlying toxicity concerns with other known methods and treatments. The compounds of the present disclosure, including H5, function as novel NME1 inhibitors that prevent the Glc-stimulated phosphorylation of histidine 58 on FAK (FAK^{pHis58}), while also functioning as new cell-cycle inhibitors that block the FAK-RB1 interaction. Targeting this pathway in ESCC tumors has not been previously reported and likely holds relevance for many glycolytic tumor types. In concert with current kinase inhibitors, FAK-targeted inhibitors are typically ATP-competitive compounds or inhibitors of scaffolding activity with signaling partners. These drugs have seen limited success. Targeting FAK^{PHis58} signaling, however, imparts an innovative approach for inhibiting the growth of ESCC tumors, tumors which have particularly evolved growth factor-independent pathways. The present drug development strategy incorporates a heretofore undescribed role for FAK^{His58} inhibitors by targeting a novel histidine phosphorylation pathway that is pivotal to ESCC proliferation, yet not induced by a growth factor, but by Glc as its sole mitogen.

### BRIEF DESCRIPTION OF THE FIGURES

For a fuller understanding of the nature and objects of the disclosure, reference should be made to the following detailed description taken in conjunction with the accompanying figures.

FIG. 1 shows antineoplastic effects of blocking glucose-induced pHis58-FAK signaling on ESCC growth. Glucose can stimulate growth factor-independent proliferation by inducing NME1 phosphorylation of FAK on His58 and FAK-RB1 interaction. FAK H58 inhibitors bind to the His58-located pocket on FAK. This blocks phosphorylation of His58 and FAK-mediated RB1 inactivation, resulting in cell cycle progession and tumor growth.

FIG. 2 shows FAK^{His58} inhibitors prevent Glc-induced ESCC proliferation. A. Top ranked small molecules that bind to FAK H58 site. B. FAK^{His58} inhibitors attenuate Glc-induced phosphorylation of FAK H58. In the absence of growth factors, KYSE70 cells were stimulated by Glc with or without FAK^{His58} inhibitors (H5). N=3, **: p<0.001 vs vehicle+Glc. C. Dose responses of FAK^{His58} inhibitors on proliferation. KYSE70 cells were incubated in serum-free medium containing Glc, BrdU and varied concentrations of FAK^{His58} inhibitors (H5). BrdU coupling ELISA was performed to assess the relative levels of newly synthesized BrdU-DNA. IC⁵⁰ values were calculated.

FIG. 3 shows Glc increases NME1 activity, and small molecules inhibit NME1-increased poHis-FAK. A. Assessment of Glc-altered NM1 phosphorylation of pHis-FAK. NME1 was pulldown with an anti-NME1 antibody from KYSE70 cells +/−Glc stimulation and added to the anti-pHis antibody (rabbit)-coated wells. The anti-FAK (mouse) was used to detect the pHis-FAK on the plate. N=3, *: p<0.05, ***: p<0.001 and ****: p<0.0001 vs No NME1 or NME1 (−Glc). B. Glc increases NME1 in xenograft lysates derived from fasting mice +/−Glc. C. H5 attenuates NME1-increased levels of pHis-rFAk. Purified NMR1 and recombinant FAK (rFAK) were incubated in the NME kinase buffer in the presence of varied concentrations (0-1 μM) of vehicle of H5 for 3 hr. The relative levels of pHis-rFAK were assessed using ELISA.

FIG. 4 shows FAK H58 inhibitors interrupt FAK-RB1 interaction. rFAK in the NME kinase buffer was incubated with varied concentrations (0-10 μM) of vehicle or H5 for 1 hr. Then, rRB1 was added to mixture and kept at room temperature for 3 hr. The mixture was added to the anti-FAK antibody-coated plated. After extensive washing, the co-IPed rRB1 was detected using an anti-RB1 antibody.

FIG. 5 shows WST1 analysis of FAK H58 inhibitor and Cisplatin-inhibited ESCC proliferation. ESCC (KYSE70) cells were seeded on a 96-well plate at a density of 2000 cells/well in complete medium. The next day, medium was replaced with the serum-reduced medium (5% FBS) containing 0-80 μM of cisplatin and 5 fixed doses of H5 (0 μM, 5 μM, 10 μM, 20 μM, or 40 μM). The cells were kept for 72 hrs. WST1 analysis, a MTT-like assay, was performed to assess proliferation. Log (inhibition) vs. response—variable (four parameters). Prism was utilized to find the best-fit value and to calculate a complete confidence interval.

FIG. 6 shows WST1 analysis of FAK H58 inhibitor and Cisplatin-inhibited ESCC proliferation. ESCC (KYSE520) cells were seeded on a 96-well plate at a density of 2000 cells/well in complete medium. The next day, medium was replaced with the serum-reduced medium (5% FBS) containing 0-80 μM of cisplatin and 5 fixed doses of H5 (0 μM, 5 μM, 10 μM, 20 μM, or 40 μM). The cells were kept for 72 hrs. WST1 analysis, a MTT-like assay, was performed to assess proliferation. Log (inhibition) vs. response—variable (four parameters). Prism was utilized to find the best-fit value and to calculate a complete confidence interval.

FIG. 7 shows WST1 analysis of FAK H58 inhibitor and Cisplatin-inhibited ESCC proliferation. ESCC (KYSE70) cells were seeded on a 96-well plate at a density of 2000 cells/well in complete medium. The next day, medium was replaced with the serum-reduced medium (5% FBS) containing 0-80 μM of cisplatin and 5 fixed doses of H5 (0 μM, 5 μM, 10 μM, 20 μM, or 40 μM). The cells were kept for 72 hrs. WST1 analysis, a MTT-like assay, was performed to assess proliferation. Log (inhibition) vs. response—variable (four parameters). Prism was utilized to find the best-fit value and to calculate a complete confidence interval.

FIG. 8 shows WST1 analysis of FAK H58 inhibitor and Cisplatin-inhibited ESCC proliferation. ESCC (KYSE520) cells were seeded on a 96-well plate at a density of 2000 cells/well in complete medium. The next day, medium was replaced with the serum-reduced medium (5% FBS) containing 0-80 μM of cisplatin and 5 fixed doses of H5 (0 μM, 5 μM, 10 μM, 20 μM, or 40 μM). The cells were kept for 72 hrs. WST1 analysis, a MTT-like assay, was performed to assess proliferation. Log (inhibition) vs. response—variable (four parameters). Prism was utilized to find the best-fit value and to calculate a complete confidence interval.

FIG. 9 shows Glc induces histidine phosphorylation of FAK. A. Nano-LC-MS analysis of FAK^{pHis58}. B. Characterization of FAK^{pHis58}. SEQ ID NO:1 is shown, where histidine is phosphorylated and threonine is either phosphorylated or not phosphorylated. C. IP/nano-LC-MS assessments of FAK^{pHis58} in KYSE70 cells +/=Glc stimulation: anti-pHis antibody (SC44-1) for IP. SEQ ID NO:2 is shown. Dark grey: high confidence and light grey: median confidence. Nano-LC-MS: samples were first reduced and alkylated by DTT and IAM, pelleted by acetone precipitation, and digested using trypsin. Derived peptides were analyzed by Nano LC-Orbitrap Lumos MS using a high-pH LC-gradient. Data analysis: generated rawfiles were searched against Homo sapiens FAK sequence and/or Homo sapiens complete protein sequence database using Sequest HT (Proteome Discoverer 1.4). Over 90% fragments of FAK were identified in the peptide fragments derived from the pHis antibody precipitates.

FIG. 10 shows Glc promotes FAK-RB1 interaction. KYSE70 cells with (+) or without (−) Glc stimulation for 1 hr were subjected to proximity ligation assays (PLA). A. PLA of Glc-induced FAK-RB1 interaction. B. H58A-attenuated, H58E-mimicked FAK-RB1 interaction. C. RB1 binding site mutation-abrogated FAK-RB1 interaction in KYSE70 cells. Duolink™ In Situ Detection PLA kit was used with anti-HA tag (mouse) and anti-RB1 (rabbit) antibodies.

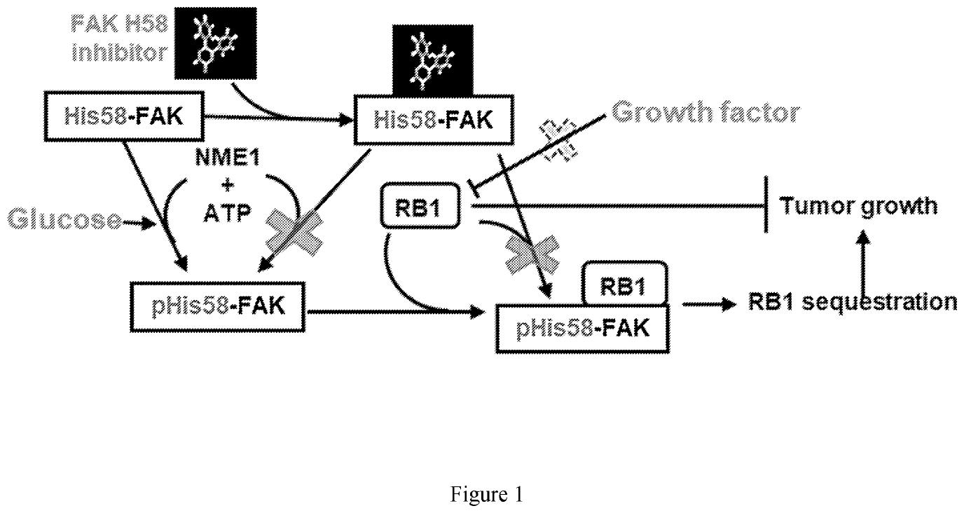

### DETAILED DESCRIPTION OF THE DISCLOSURE

Although the subject matter will be described in terms of certain examples, other examples, including examples that do not provide all of the benefits and features set forth herein, are also within the scope of this disclosure. Various structural, logical, and process steps may be made without departing from the scope of the disclosure.

The present disclosure provides compounds and compositions that inhibit glucose-induced growth signaling. The compounds may be suitable to treat glycolytic cancers, such as, for example, esophageal squamous cell carcinoma (ESCC). The compounds may be used to inhibit or partially inhibit glucose-promoted tumor cell proliferation, NME-1 catalyzed histidine phosphorylation of FAK, and FAK interaction with RB1

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

As used herein, unless otherwise indicated, the term “group” refers to a chemical entity that is monovalent (i.e., has one terminus that can be covalently bonded to other chemical species as in a methyl or phenyl group), divalent, or polyvalent (i.e., has two or more termini that can be covalently bonded to other chemical species as in a methylene or phenylene group). The term “group” also includes radicals (e.g., monovalent radicals and multivalent radicals, such as, for example, divalent radicals, trivalent radicals, and the like).

As used herein, unless otherwise indicated, the term “aliphatic” refers to branched or unbranched hydrocarbon groups that, optionally, contain one or more degree(s) of unsaturation. Degrees of unsaturation can arise from, but are not limited to, cyclic aliphatic groups. For example, the aliphatic groups/moieties are a C₁ to C₁₂ aliphatic group, including all integer numbers of carbons and ranges of numbers of carbons therebetween (e.g., C₁, C₂, C₃, C₄, C₅, C₆, C₇, C₈, C₉, C₁₀, C₁₁, and C₁₂). Aliphatic groups include, but are not limited to, alkyl groups (e.g., methyl, ethyl, propyl, isopropyl, butyl, n-butyl, t-butyl, sec-butyl, isobutyl, n-pentyl, tert-pentyl, neopentyl, isopentyl, sec-pentyl, 3-pentyl, sec-isopentyl, active pentyl, and the like), alkenyl groups, and alkynyl groups. The aliphatic group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, phosphate groups, phosphonate groups, thioether groups, thioester groups, and the like, and combinations thereof.

As used herein, unless otherwise indicated, the term “alkyl group” refers to branched or unbranched saturated hydrocarbon groups. Examples of alkyl groups include, but are not limited to, methyl groups, ethyl groups, n- and isopropyl groups, n-, sec-, iso- and tert-butyl groups, and the like. The alkyl group can be a C₁ to C₁₂ alkyl group, including all integer numbers of carbons and ranges of numbers of carbons there between (e.g., C₁, C₂, C₃, C₄, C₅, C₆, C₇, C₈, C₉, C₁₀, C₁₁, or C₁₂). The alkyl group can be unsubstituted or substituted with one or more substituents. Examples of substituents include, but are not limited to, various substituents such as, for example, halogens (e.g., —F, —Cl, —Br, and —I), aliphatic groups (e.g., alkyl groups, alkenyl groups, and alkynyl groups), aryl groups, alkoxide groups, carboxylate groups, carboxylic acids, ether groups, alcohol groups, amine groups, thiol groups, thioether groups, and the like, and combinations thereof.

As used herein, unless otherwise indicated, the term “heteroalkyl group” refers to branched or unbranched, saturated or unsaturated hydrocarbon groups comprising at least one heteroatom. Examples of suitable heteroatoms include, but are not limited to, nitrogen, oxygen, sulfur, phosphorus, and the halogens. The heteroalkyl group can be unsubstituted or substituted with one or more substituents. Examples of substituents include, but are not limited to, various substituents such as, for example, halogens (e.g., —F, —Cl, —Br, and —I), aliphatic groups (e.g., alkyl groups, alkenyl groups, and alkynyl groups), aryl groups, alkoxide groups, carboxylate groups, carboxylic acids, ether groups, alcohol groups, amine groups, thiol groups, thioether groups, and the like, and combinations thereof.

As used herein, unless otherwise indicated, the term “aryl group” refers to C₅ to C₁₂ aromatic or partially aromatic carbocyclic groups, including all integer numbers of carbons and ranges of numbers of carbons therebetween (e.g., C₅, C₆, C₇, C₈, C₉, C₁₀, C₁₁, or C₁₂). An aryl group can also be referred to as an aromatic group. The aryl groups can comprise polyaryl groups such as, for example, fused ring or biaryl groups. The aryl group can be unsubstituted or substituted with one or more substituent. Examples of substituents include, but are not limited to, substituents such as, for example, halogens (e.g., —F, —Cl, —Br, and —I), aliphatic groups (e.g., alkyl groups, alkenyl groups, and alkynyl groups), aryl groups, alkoxide groups, carboxylate groups, carboxylic acids, ether groups, alcohol groups, amine groups, thiol groups, thioether groups, and the like, and combinations thereof. Examples of aryl groups include, but are not limited to, phenyl groups, biaryl groups (e.g., biphenyl groups and the like), and fused ring groups (e.g., naphthyl groups and the like).

As used herein, unless otherwise indicated, the terms “carbocyclic” or “heterocyclic” means a carbon-containing ring or a carbon-containing ring in which one or more of the carbon atoms are replaced by a heteroatom, respectively. These groups may be non-aromatic or aromatic. Carbocyclic or heterocyclic groups may be saturated or unsaturated and may have one or more substituent (e.g., hydroxy, alkoxy, thioalkoxy, halogens, and the like), and combinations thereof. Additional examples of substituents include, but are not limited to, halogens (e.g., —F, —Cl, —Br, and —I), aliphatic groups (e.g., alkyl groups, alkenyl groups, and alkynyl groups), aryl groups, alkoxide groups, carboxylate groups, carboxylic acids, ether groups, alcohol groups, amine groups, thiol groups, thioether groups, and the like, and combinations thereof.

As used herein, unless otherwise indicated, the term “heterocyclic group” refers to C₃-C₂₀ cyclic groups containing one or more heteroatoms (e.g., N, O, S, or the like) as part of the ring structure, including all integer numbers of carbons and ranges of numbers of carbons therebetween (C₃, C₄, C5, C₆, C₇, C₈, C₉, C₁₀, C₁₁, C₁₂, C₁₃, C₁₄, C₁₅, C₁₆, C₁₇, C₁₈, C₁₉, or C₂₀). The heterocyclic groups may be substituted or unsubstituted and/or have additional degrees of unsaturation. Examples of substituents include, but are not limited to, halogens (e.g., —F, —Cl, —Br, and —I), aliphatic groups (e.g., alkyl groups, alkenyl groups, and alkynyl groups), aryl groups, alkoxide groups, carboxylate groups, carboxylic acids, ether groups, alcohol groups, amine groups, thiol groups, thioether groups, and the like, and combinations thereof. The heterocyclic groups can be fused to carbocyclic groups or to each other. Non-limiting examples of heterocyclic groups include furanyl groups, oxazolyl groups, isothiazolyl groups, thiazolyl groups, tetrahydropyranyl groups, piperazinyl groups, dioxanyl groups, pyrrolidinyl groups, tetrahydrothiophenyl groups, tetrahydrofuranyl groups, quinuclidinyl groups, azaadamantanyl groups, decahydroquinolinyl groups, and the like.

As used herein, unless otherwise indicated, the term “heteroaryl group” means a monovalent monocyclic or polycyclic aromatic group of 5 to 18 ring atoms or a polycyclic aromatic group, containing one or more ring heteroatoms selected from N, O, or S, the remaining ring atoms being C, including all integer number of ring atoms and ranges therebetween (e.g., 5, 6, 7, 8, 9, 10, 11, 12, 13, 14, 15, 16, 17, or 18). Heteroaryl as herein defined also means a polycyclic (e.g., bicyclic) heteroaromatic group where the heteroatom is selected from N, O, or S. The aromatic radical is optionally substituted independently with one or more substituents described herein. The substituents can themselves be optionally substituted. Examples of substituents include, but are not limited to, halogens (e.g., —F, —Cl, —Br, and —I), aliphatic groups (e.g., alkyl groups, alkenyl groups, and alkynyl groups), aryl groups, alkoxide groups, carboxylate groups, carboxylic acids, ether groups, alcohol groups, amine groups, thiol groups, thioether groups, and the like, and combinations thereof. Examples of heteroaryl groups include, but are not limited to, benzothienyl, furyl, thienyl, pyrrolyl, pyridyl, pyrazinyl, pyrazolyl, pyridazinyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, benzoimidazolyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1λ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo [1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two fused rings the heteroaryl groups herein defined may have an unsaturated or partially saturated ring fused with a fully saturated ring.

In an aspect, the present disclosure provides compounds. The compounds may be used treat someone having or suspected of having cancer (e.g., esophageal squamous cell carcinoma (ESCC)). The compounds of the present disclosure may be used to as NME1 inhibitors that prevent glucose-stimulated phosphorylation of histidine 58 on FAK.

In various examples, a compound of the present disclosure has the following structure:

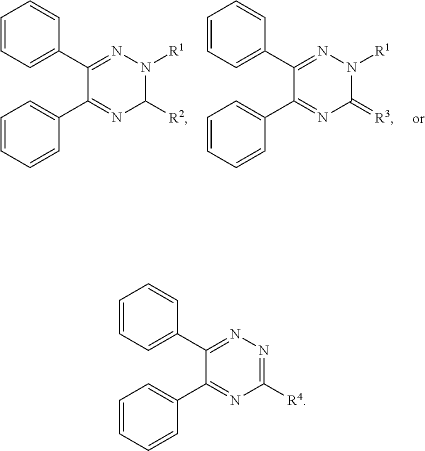

R¹ is chosen from H, substituted or unsubstituted aliphatic groups, and substituted or unsubstituted aryl groups. R³ is a double bonded heteroatom (e.g., S or O). R⁴ is a substituted or unsubstituted alkyl group or substituted or unsubstituted heteroalkyl group. R¹ and R² are combined to form a substituted or unsubstituted carbocyclic ring, a substituted or unsubstituted heterocyclic ring, a substituted or unsubstituted aryl ring, or a substituted or unsubstituted heteroaryl ring or R¹ and R³ are combined to form a substituted or unsubstituted carbocyclic ring, a substituted or unsubstituted heterocyclic ring, a substituted or unsubstituted aryl ring, or a substituted or unsubstituted heteroaryl ring. Examples of aliphatic groups, alkyl groups, aryl groups, heteroalkyl groups, carbocyclic groups, and heterocyclic groups are provided herein.

In various examples, R¹ and R² or R¹ and R³ combine to form a substituted or unsubstituted 5-membered carbocyclic, heterocyclic, or heteroaryl ring; substituted or unsubstituted 6-membered carbocyclic, heterocyclic, aryl, or heteroaryl ring, or a substituted or unsubstituted 7-membered carbocyclic or heterocyclic ring.

The R¹ groups, R⁴ groups, and rings formed from R¹ and R² or R¹ and R³ may have various substituents. Examples of substituents include, but are not limited to, substituents such as, for example, halogens (e.g., —F, —Cl, —Br, and —I), aliphatic groups (e.g., alkyl groups, alkenyl groups, and alkynyl groups), aryl groups, alkoxide groups, carboxylate groups, carboxylic acids, ether groups, alcohol groups, amine groups, thiol groups, thioether groups, and the like, and combinations thereof.

In various examples, a compound of the present disclosure has the following structure:

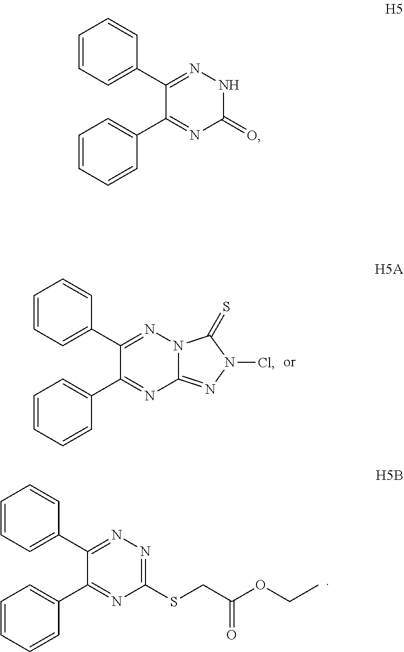 [SMILES: O=c1nc(-c2ccccc2)c(-c2ccccc2)n[nH]1 ; S=c1n(Cl)nc2nc(-c3ccccc3)c(-c3ccccc3)nn12 ; CCOC(=O)CSc1nnc(-c2ccccc2)c(-c2ccccc2)n1]

Additional examples of compounds of the present disclosure include, but are not limited to,

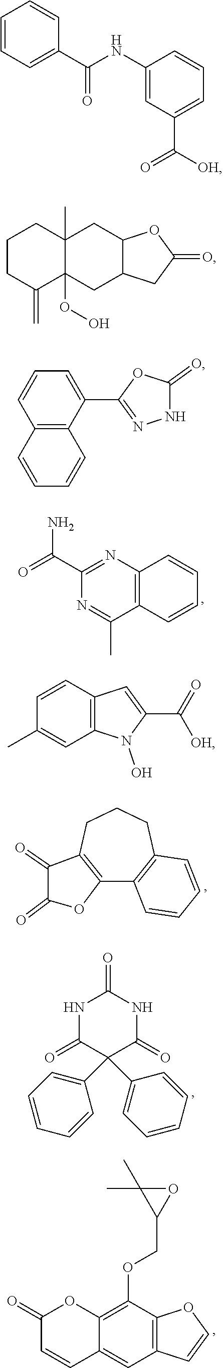

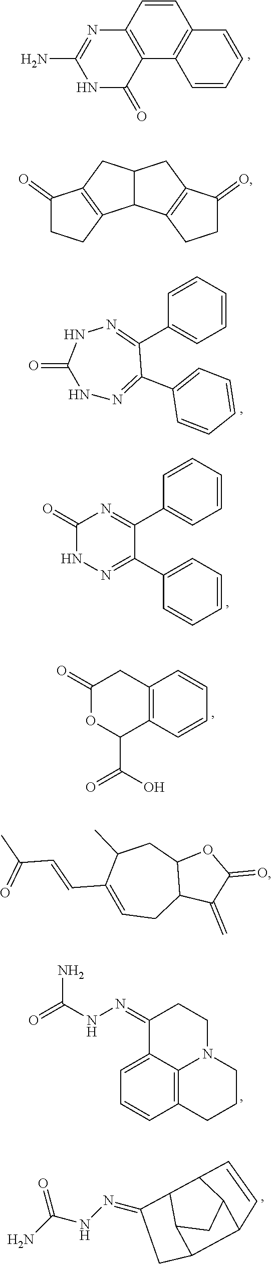

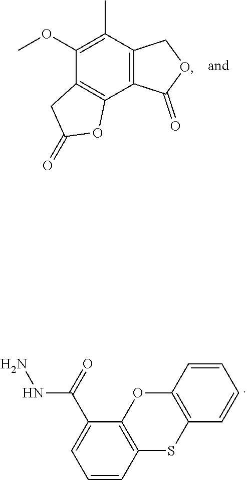 [SMILES: COc1c(C)c2c(c3c1CC(=O)O3)C(=O)OC2 ; NNC(=O)c1cccc2c1Oc1ccccc1S2]

The compounds of the present disclosure may bind to the FAK^{His58} site. After binding to the FAK^{His58} site, glucose-promoted tumor cell proliferation, NME-1 catalyzed histine phosphorylation of FAK, and FAK interaction with RB1 is inhibited or partially inhibited.

In an aspect, the present disclosure provides compositions comprising compounds of the present disclosure. The compositions further comprise one or more pharmaceutically acceptable carrier.

A composition may comprise additional components. For example, the composition comprises a buffer solution suitable for administration to an individual (e.g., a mammal such as, for example, a human or a non-human). An individual may be a subject. The buffer solution may be a pharmaceutically acceptable carrier.

The composition of the disclosure may also be formulated into a sterile solid preparation, for example, by freeze-drying, and can be used after sterilized or dissolved in sterile injectable water or other sterile diluent(s) immediately before use. Additional examples of pharmaceutically acceptable carriers include, but are not limited to, sugars, such as lactose, glucose, and sucrose; starches, such as corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations; and combinations thereof. Additional non-limiting examples of pharmaceutically acceptable carriers can be found in: Remington: The Science and Practice of Pharmacy (2012) 22nd Edition, Philadelphia, PA. Lippincott Williams & Wilkins.

In various examples, one or more compounds and/or one or more compositions comprising one or more compounds described herein are be administered to a subject in need of treatment using any known method and route, including oral, parenteral, subcutaneous, intraperitoneal, intrapulmonary, intranasal and intracranial injections. Parenteral infusions include intramuscular, intravenous, intraarterial, intraperitoneal, and subcutaneous administration. Topical and/or transdermal administrations are also encompassed.

In an aspect, the present disclosure provides methods of using one or more compound or composition thereof. One or more compounds of the present disclosure or a composition of the present disclosure can be used to treat cancer. Methods of the present disclosure may be used to inhibit cell growth of malignant cells and/or hyperplastic cells. In various examples, a method of the present disclosure may be used to block glucose-induced growth signaling, but not glucose (Glc) uptake/metabolism. A method can be carried out in combination with one or more known therapies.

Non-limiting examples of cancers include glycolytic cancers (e.g., glycolytic tumors). Examples of glycolytic cancers include, but are not limited to, esophageal carcinoma (e.g., esophageal squamous cell carcinoma (ESCC) and drug-resistant ESCC), and carcinoma on other parts of the body including the lung, mucous membranes, and urinary tract that have cancerous squamous cells with Glc-induced growth signaling that could be treated in a manner similar to ESCC. Reference to ESCC includes drug-resistant ESCC.

A method of the present disclosure may be used to treat ESCC by inhibiting Glc-induced growth signaling, while not inhibiting Glc uptake/metabolism. Without intending to be bound by any particular theory, it is considered that a method of the present disclosure overcomes the obstacles current clinical therapies that target receptor tyrosine kinases and avoid underlying toxicity concerns associated with those therapies.

Compounds of the present disclosure may be used in a method for treating diseases associated with malignant cells (for example, ESCC). For example, the method inhibit or partially inhibit glucose-promoted tumor cell proliferation, NME-1 catalyzed histidine phosphorylation of FAK, and FAK interaction with RB1.

A method may be carried out in a subject in need of treatment who has been diagnosed with or is suspected of having ESCC or drug-resistant ESCC. A method may also be carried out in a subject who have a relapse or a high risk of relapse after being treated for ESCC. The subject may be referred to as an individual.

A method of treating a disease (e.g., cancer, such as, for example, a glycolytic cancer, such as, for example, ESCC) comprises administering to a subject in need of a treatment (e.g., an individual in need of treatment) a therapeutic amount (e.g., an amount of compound (e.g., a compound having the following structure:

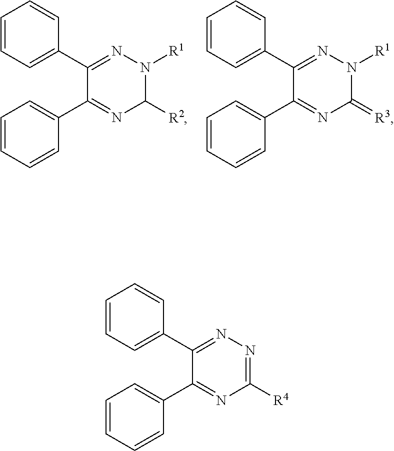

or a combination thereof, or a compound having the following structure:

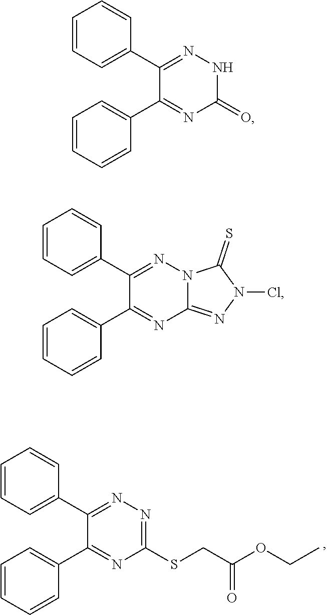 [SMILES: O=c1nc(-c2ccccc2)c(-c2ccccc2)n[nH]1 ; S=c1n(Cl)nc2nc(-c3ccccc3)c(-c3ccccc3)nn12 ; CCOC(=O)CSc1nnc(-c2ccccc2)c(-c2ccccc2)n1]

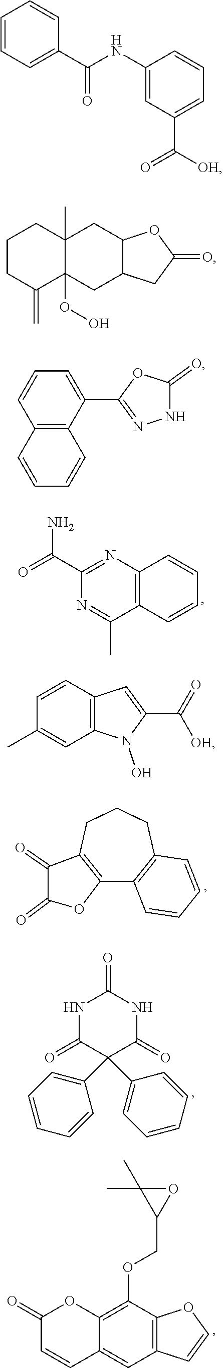

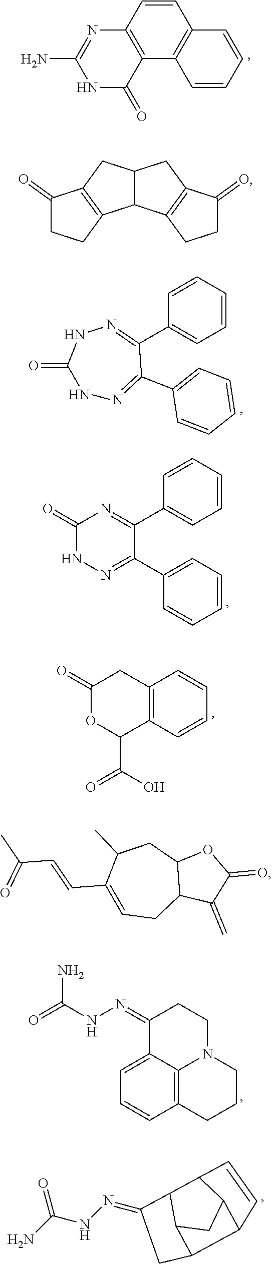

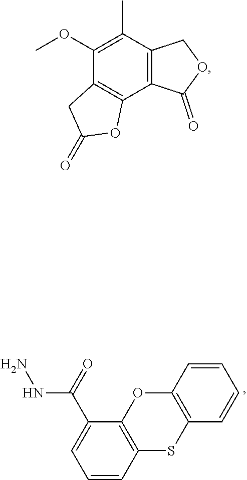 [SMILES: COc1c(C)c2c(c3c1CC(=O)O3)C(=O)OC2 ; NNC(=O)c1cccc2c1Oc1ccccc1S2]

or a combination thereof) or composition sufficient to treat the subject) of a compound or composition of the present disclosure, where the subject's disease is treated.

In various examples, a compound of the present disclosure is used to inhibit the growth of cells (e.g., malignant cells, such as, for example, cancer cells, such as, for example, cancer cells associated with ESCC). For example, growth of cancer cells (e.g., cells associated with ESCC) is inhibited by contacting the cancer cells with a compound in an amount (e.g., 1 nM to 1 mM) and time sufficient to cause binding to the FAK^{His58} site and inhibit or partially inhibit glucose-promoted tumor cell proliferation, NME-1 catalyzed histine phosphorylation of FAK, and FAK interaction with RB1. Inhibition of cell growth refers to any decrease in growth/reproduction of a cell (e.g., the growth/reproduction of cancer cells). The method may also be a method to reduce the size of a tumor.

A method of inhibiting cell proliferation comprises contacting a cell with a compound of the present disclosure or a composition comprising a compound of the present disclosure.

In various examples, a subject in need of treatment is administered a therapeutically effective amount of a compound in a composition of the present disclosure. A dose of a therapeutically effective amount of a compound of the present disclosure may have a concentration of 1 nM to 10 mM, including all 0.1 nM values and ranges therebetween. In an example, a dose of a therapeutically effective amount of a compound in a composition of the present disclosure may have a concentration of 1-500 μM, 50-500 μM, 1-250 μM, 10-250 μM, 25-250 μM, 25-150 μM, 50-250 μM, or 50-150 μM.

In an example, an individual in need of treatment is administered a compound or a composition comprising the compound of the present disclosure in multiple doses dose (e.g., multiple administration steps). Following the multiple doses, the individual's mitochondrial unfolded protein response activity is ameliorated for 1-120 hours (e.g., 24-120 hours, 1-48 hours, 12-48 hours, or 24-48 hours), including all second values and ranges therebetween.

A method of this disclosure may be carried out in combination with one or more known therapy(ies), including, but not limited to, surgery, radiation therapy, chemotherapy, photodynamic therapy, and/or immunotherapy.

In various examples, the composition of the present disclosure may be administered in combination with one or more chemotherapy drugs. The composition may be administered sequentially or concurrently with one or more chemotherapy drugs. The sequential administration of the composition and one or more chemotherapy drugs may be separated by seconds, minutes, hours, days, or weeks. Examples of chemotherapy drugs that may be used in combination with the composition include, but are not limited to oxaliplatin, 5-FU, paclitaxel, cisplatin, carboplatin, and the like, and combinations thereof. For example, using a composition comprising a compound of the present disclosure (e.g., a compound having the following structure:

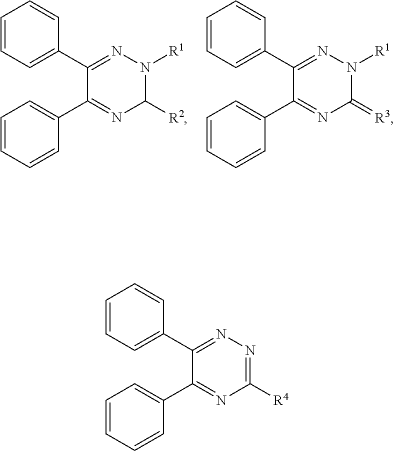

or a combination thereof, or a compound having the following structure:

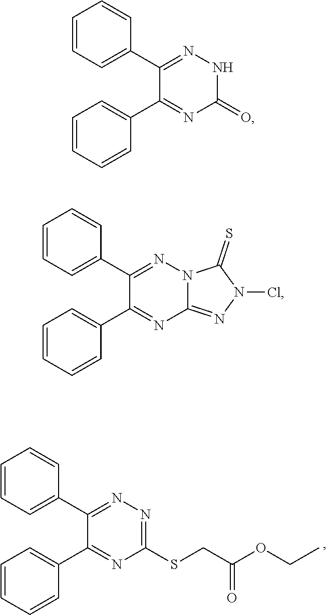 [SMILES: O=c1nc(-c2ccccc2)c(-c2ccccc2)n[nH]1 ; S=c1n(Cl)nc2nc(-c3ccccc3)c(-c3ccccc3)nn12 ; CCOC(=O)CSc1nnc(-c2ccccc2)c(-c2ccccc2)n1]

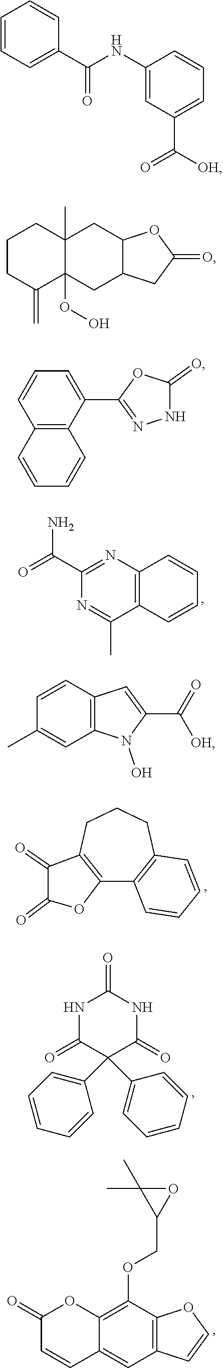

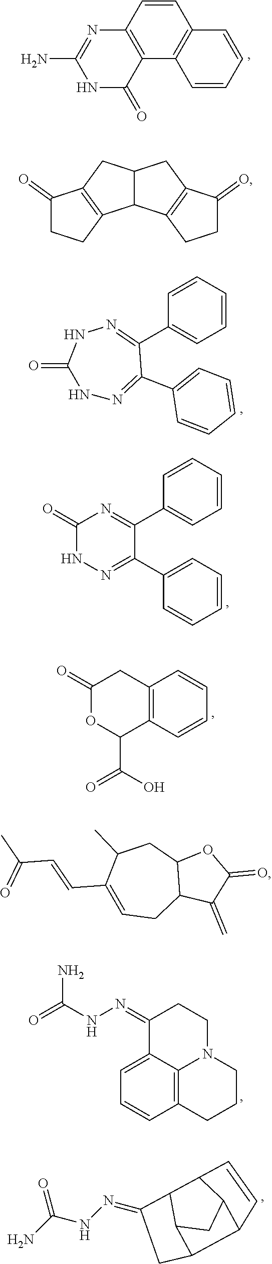

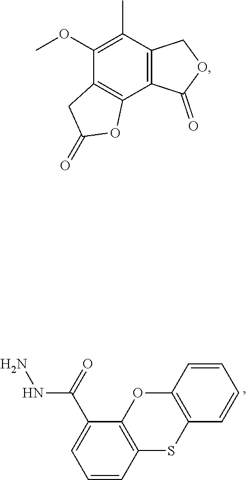 [SMILES: COc1c(C)c2c(c3c1CC(=O)O3)C(=O)OC2 ; NNC(=O)c1cccc2c1Oc1ccccc1S2]

or a combination thereof) in combination with one or more chemotherapy drugs may increase the efficacy of the one or more chemotherapy drugs (e.g., oxaliplatin, 5-FU, paclitaxel, cisplatin, carboplatin, or the like, or a combination thereof).

A subject in need of treatment or individual in need of treatment may be a human or non-human mammal. Non-limiting examples of non-human mammals include cows, pigs, mice, rats, rabbits, cats, dogs, or other agricultural animals, pets, service animals, and the like.

In an aspect, the disclosure provides kits. In various examples, a kit comprises a pharmaceutical preparation containing any one or any combination of compounds of the present disclosure. In an example, the instant disclosure includes a closed or sealed package that contains the pharmaceutical preparation. In various examples, the package comprises one or more closed or sealed vials, bottles, blister (bubble) packs, or any other suitable packaging for the sale, distribution, or use of the pharmaceutical compounds and compositions comprising them. The printed material may include printed information. The printed information may be provided on a label, on a paper insert, or printed on packaging material. The printed information may include information that identifies the compound in the package, the amounts and types of other active and/or inactive ingredients in the composition, and instructions for taking the compound and/or composition. The instructions may include information, such as, for example, the number of doses to take over a given period of time, and/or information directed to a pharmacist and/or another health care provider, such as a physician, or a patient. The printed material may include an indication that the pharmaceutical composition and/or any other agent provided therein is for treatment of a subject having cancer (e.g., glycolytic cancers, such as, for example, ESCC). In various examples, the kit includes a label describing the contents of the kit and providing indications and/or instructions regarding use of the contents of the kit to treat a subject having any cancer and/or other diseases.

In various examples, kits comprise materials that can be used for administration to individuals in need of ESCC treatment. A kit, for example, can comprise one or more therapeutics that may be in a lyophilized form, optionally reconstitution media, and instructions for administration. A kit can comprise a single dose or multiple doses.

The steps of the method described in the various embodiments and examples disclosed herein are sufficient to carry out the methods of the present disclosure. The methods described in the embodiment are a combination of steps of the disclosed methods. In another embodiment, the method consists of such steps.

The following Statements provide examples of compounds of the present disclosure, methods using compounds of the present disclosure, and uses of compounds of the present disclosure.

Statement 1. A compound having the structure:

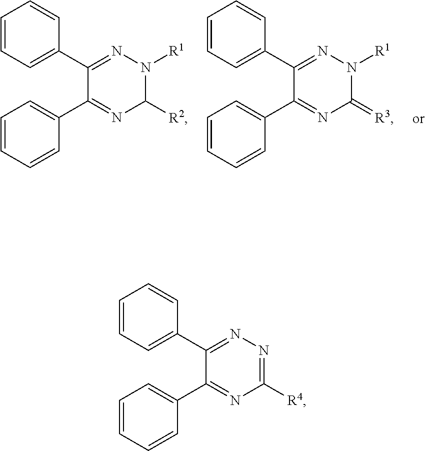

wherein R¹ is chosen from H, substituted or unsubstituted aliphatic groups, and substituted or unsubstituted aryl groups; R³ is a double bonded heteroatom (e.g., S or O); R⁴ is a substituted or unsubstituted alkyl group or substituted or unsubstituted heteroalkyl group; or R¹ and R² are combined to form a substituted or unsubstituted carbocyclic ring, a substituted or unsubstituted heterocyclic ring, a substituted or unsubstituted aryl ring, or a substituted or unsubstituted heteroaryl ring; or R¹ and R³ are combined to form a substituted or unsubstituted carbocyclic ring, a substituted or unsubstituted heterocyclic ring, a substituted or unsubstituted aryl ring, or a substituted or unsubstituted heteroaryl ring.
Statement 2. A compound of Statement 1, having the following structure:

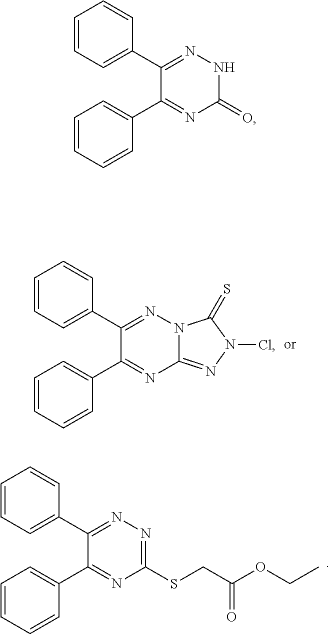 [SMILES: O=c1nc(-c2ccccc2)c(-c2ccccc2)n[nH]1 ; S=c1n(Cl)nc2nc(-c3ccccc3)c(-c3ccccc3)nn12 ; CCOC(=O)CSc1nnc(-c2ccccc2)c(-c2ccccc2)n1]

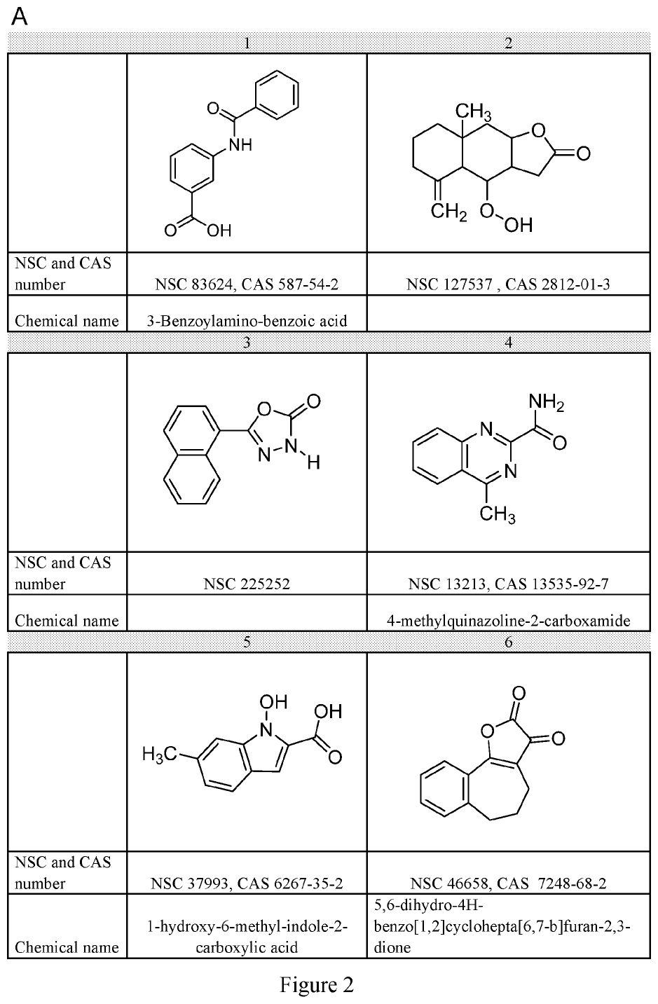

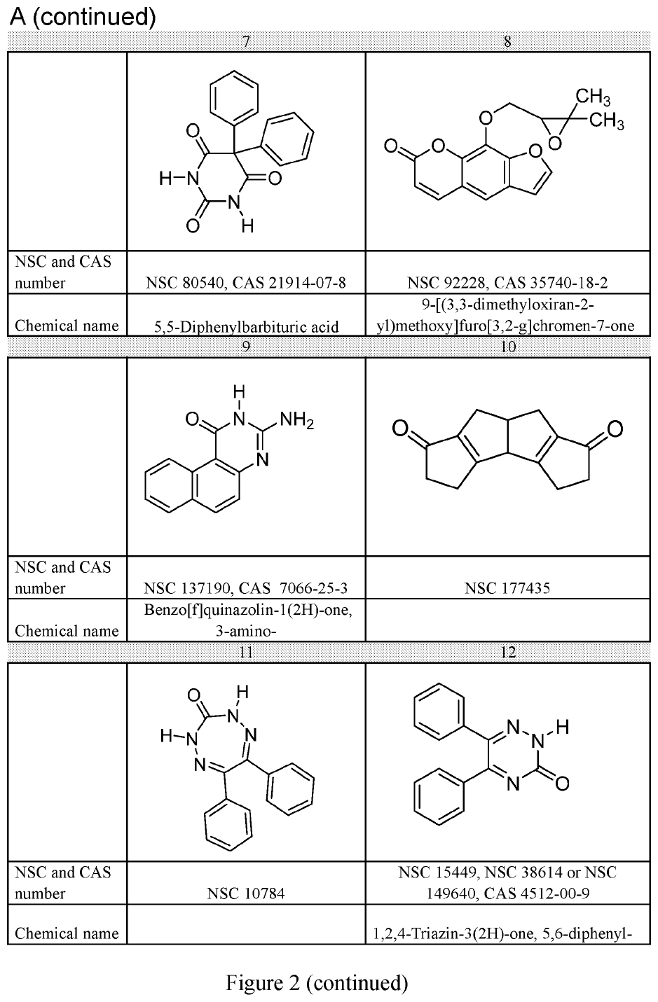

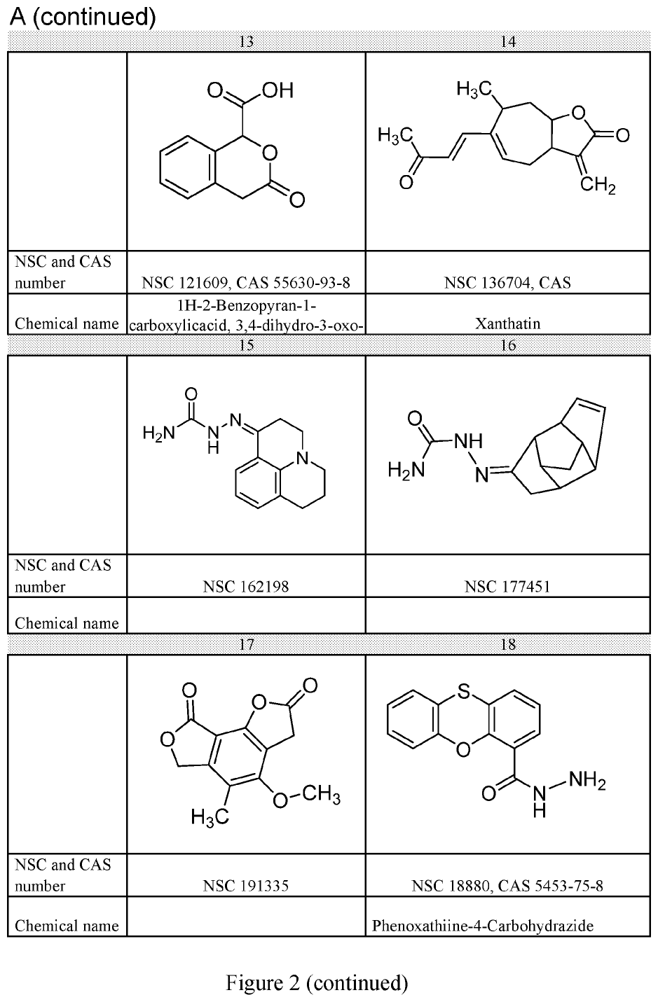

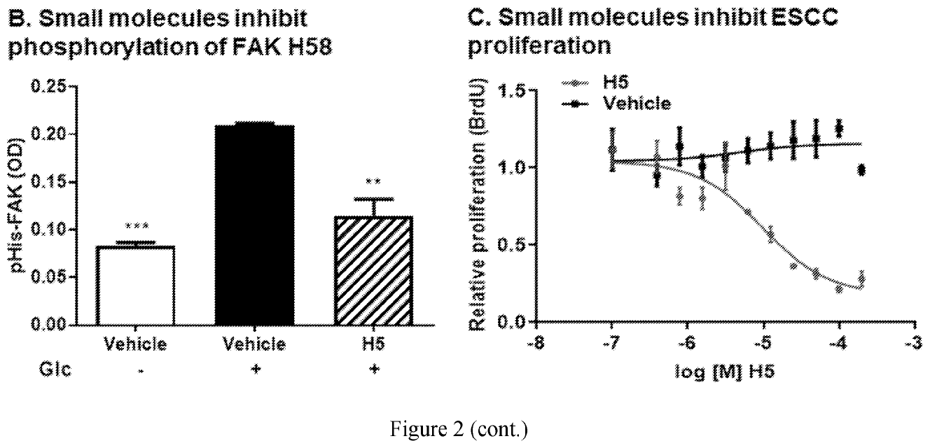

Statement 3. A method for treating an individual having cancer (e.g., drug-resistant esophageal squamous cell carcinoma) or suspected of having cancer (e.g., drug-resistant esophageal squamous cell carcinoma), comprising administering to the individual a compound of the present disclosure (e.g., a compound of Statements 1 or 2) or a composition comprising a compound of the present disclosure (e.g., a compound of Statements 1 or 2).
Statement 4. A compound having the structure of a compound of FIG. 2A.
Statement 5. A compound having the structure:

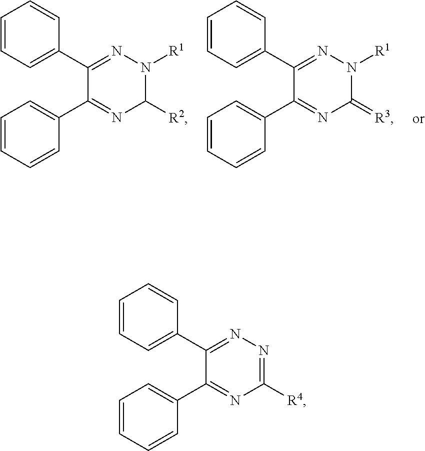

wherein R¹ is chosen from H, substituted or unsubstituted aliphatic groups, and substituted or unsubstituted aryl groups; R³ is a double bonded S or O; R⁴ is a substituted or unsubstituted alkyl group or substituted or unsubstituted heteroalkyl group; or R¹ and R² are combined to form a substituted or unsubstituted carbocyclic ring, a substituted or unsubstituted heterocyclic ring, a substituted or unsubstituted aryl ring, or a substituted or unsubstituted heteroaryl ring; or R¹ and R³ are combined to form a substituted or unsubstituted carbocyclic ring, a substituted or unsubstituted heterocyclic ring, a substituted or unsubstituted aryl ring, or a substituted or unsubstituted heteroaryl ring.
Statement 6. A compound of Statement 5, wherein R¹ and R² or R¹ and R³ combine to form a substituted or unsubstituted 5-membered carbocyclic, heterocyclic, or heteroaryl ring; substituted or unsubstituted 6-membered carbocyclic, heterocyclic, aryl, or heteroaryl ring, or a substituted or unsubstituted 7-membered carbocyclic or heterocyclic ring.
Statement 7. The compound of any one of Statements 5 or 6, having the following structure:

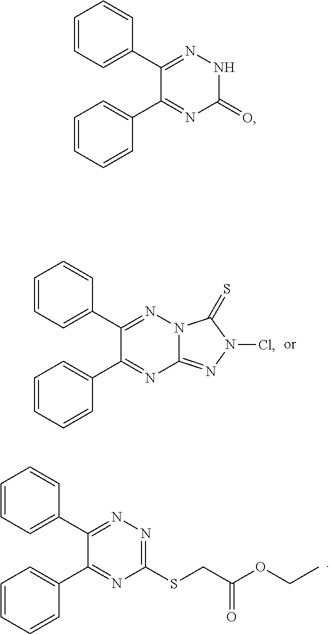 [SMILES: O=c1nc(-c2ccccc2)c(-c2ccccc2)n[nH]1 ; S=c1n(Cl)nc2nc(-c3ccccc3)c(-c3ccccc3)nn12 ; CCOC(=O)CSc1nnc(-c2ccccc2)c(-c2ccccc2)n1]

Statement 8. A composition comprising a compound of any one of Statements 5-7 and a pharmaceutically acceptable carrier.
Statement 9. A composition of Statement 8, wherein the compound has the following structure:

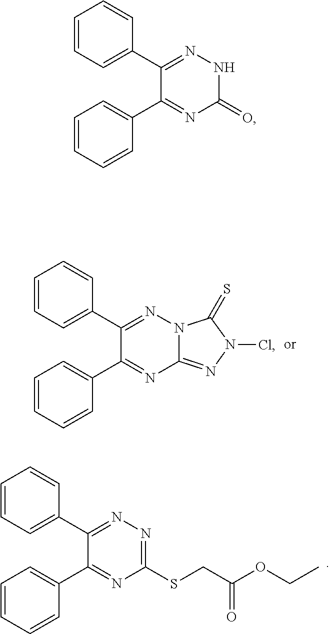 [SMILES: O=c1nc(-c2ccccc2)c(-c2ccccc2)n[nH]1 ; S=c1n(Cl)nc2nc(-c3ccccc3)c(-c3ccccc3)nn12 ; CCOC(=O)CSc1nnc(-c2ccccc2)c(-c2ccccc2)n1]

Statement 10. A method for treating an individual having cancer or suspected of having cancer, comprising administering to the individual a therapeutically effective amount compound of any one of Statements 5-8 or a composition of Statements 8 or 9.
Statement 11. A method of Statement 10, wherein the cancer is a carcinoma of the lung, mucous membranes, or urinary tract, wherein the carcinoma of lung, mucous membranes, or urinary tract have cancerous squamous cells with Glc-induced growth signaling.
Statement 12. A method of Statement 10, wherein the cancer is drug-resistant esophageal squamous cell carcinoma or esophageal squamous cell carcinoma.
Statement 13. A method of any one of Statements 10-12, wherein the compound has the following structure:

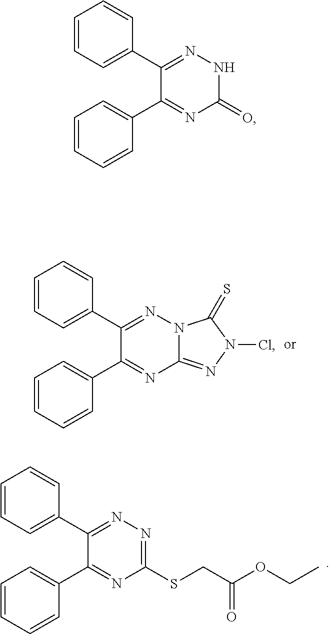 [SMILES: O=c1nc(-c2ccccc2)c(-c2ccccc2)n[nH]1 ; S=c1n(Cl)nc2nc(-c3ccccc3)c(-c3ccccc3)nn12 ; CCOC(=O)CSc1nnc(-c2ccccc2)c(-c2ccccc2)n1]

Statement 14. A method of any one of Statements 10-13, further comprising (e.g., performing or administering) surgery, radiation therapy, chemotherapy, photodynamic therapy, and/or immunotherapy.
Statement 15. A compound having the following structure:

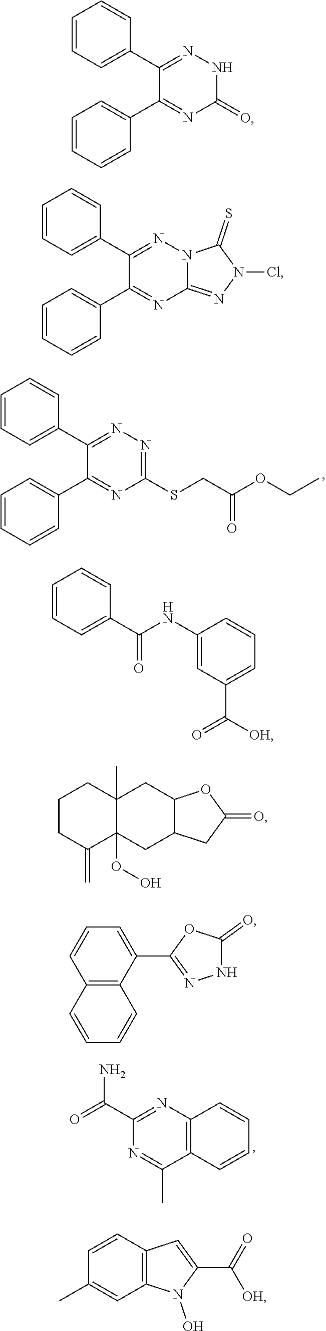

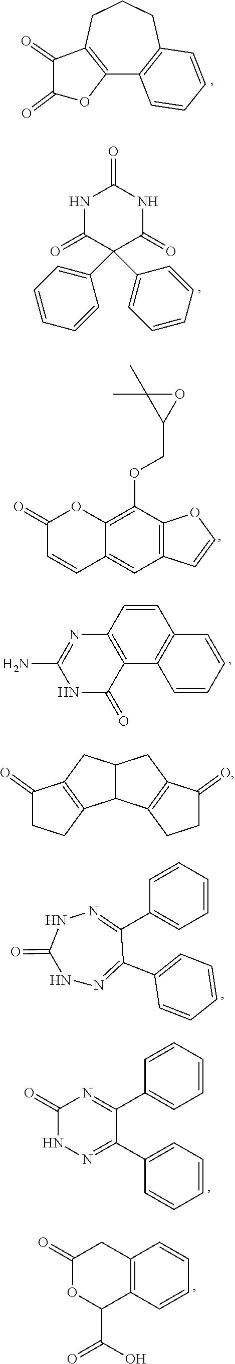

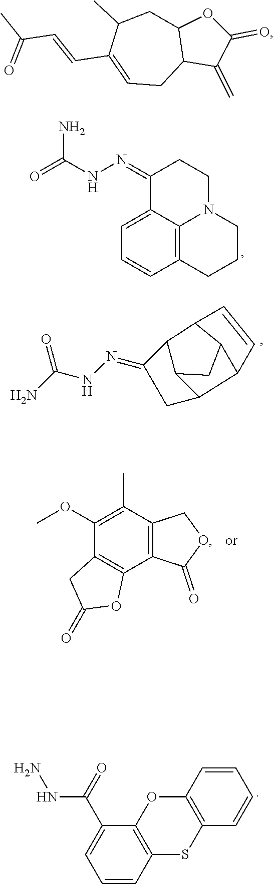

Statement 16. A composition comprising a compound of Statement 15 and a pharmaceutically acceptable carrier.
Statement 17. A method for treating an individual having cancer or suspected of having cancer, comprising administering to the individual a therapeutically effective amount compound of Statement 15 or a composition of Statement 16.
Statement 18. A method of Statement 17, wherein the cancer is a carcinoma of the lung, mucous membranes, or urinary tract, wherein the carcinoma of lung, mucous membranes, or urinary tract have cancerous squamous cells with Glc-induced growth signaling.
Statement 19. A method of Statement 17, wherein the cancer is drug-resistant esophageal squamous cell carcinoma or esophageal squamous cell carcinoma.
Statement 20. A method of anyone of Statements 17-19, further comprising (e.g., performing or administering) surgery, radiation therapy, chemotherapy, photodynamic therapy, and/or immunotherapy.
Statement 21. A method for inhibiting glucose-induced growth signaling, comprising administering a therapeutically effective amount of a compound of any one of Statements 5-7 or a composition of Statements 8 or 9, wherein glucose uptake or metabolism is not inhibited.
Statement 22. A method for inhibiting glucose-induced growth signaling, comprising administering a therapeutically effective amount of a compound of Statement 15 or a composition of Statement 16, wherein glucose uptake or metabolism is not inhibited.

The following example is presented to illustrate the present disclosure. It is not intended to be limiting in any matter.

### Example

This example provides a description of compounds of the present disclosure.

Using a small molecule paradigm, the compounds of the present disclosure target and exploit the tumor proliferation mechanism employed by cancers refractory to growth factor inhibition (GFI) therapies, e.g., drug-resistant esophageal squamous cell carcinoma (ESCC). In these cases, the tumor escapes dependence on cellular growth factors, and consequently do not succumb to GFI treatments inasmuch as they are able to reprogram glucose metabolism to promote tumor growth. Glucose-induced proliferation is initiated by histidine kinases with modifications of focal adhesion kinase (FAK) on its histidine amino acids. Histidine-phosphorylation (pHis) of FAK promotes FAK interaction with and sequestration of RB1, leading to cell cycle progression. Novel small molecules that target the tumor's dependence on glucose induced FAK activity, therefore, prevent tumor progression in patients possessing malignancies refractory to GFI therapy or that have an overactivated glucose metabolism.

Using structure-based virtual high-throughput screening on FAK^{His58}, active hits were identified that have been selected to bind to the FAK^{His58} site using molecular modeling methods with available crystal structures in the PDB, Zinc 15, and NCI ligand databases. Cellular and biochemical studies indicate that selected hits inhibited Glc-promoted tumor cell proliferation, NME1-catalyzed histidine phosphorylation of FAK, and FAK interaction with RB1. Activity, targeting Glc-stimulated pHis signaling and ESCC proliferation, has been observed in the high nM to low μM concentrations. It was reported that His58 was a major pHis site on FAK, which therefore plays a key role in Glc-induced ESCC proliferation.

Molecular modeling. Active hits that bind to FAK^{His58} are shown in FIG. 2A. A representative small molecule (H5) decreased Glc-induced FAK^{pHis58} as shown in FIG. 2B. In addition, hits caused a dose dependent reduction in ESCC proliferation as shown in FIG. 2C. The IC50 values for the hits are recited per Table 1.

TABLE 1
IC⁵⁰ of representative hits. The effects of FAK H58 inhibitors on
ESCC cell proliferation. ESCC cells were incubated in serum-free
medium containing Glc, BrdU and varied concentrations of FAK H58
inhibitors and subjected to ELISA. IC⁵⁰ values were calculated.

| FAK H58 Inhibitor | H5 | H20 | H37 | H38 |
|---|---|---|---|---|
| IC⁵⁰ (μM) | 9 | 41 | 36 | 64 |

Preparation of the ligand sets: The NCI database was downloaded, compounds with alias groups were removed. Molecular properties were calculated with Chemaxon Jchem software for each molecule, namely log P, PSA, number of hydrogen bond donors and acceptors and number of rotatable bonds. The compound set was filtered for lead likeness as previously described. That is, log P less than 3, molecular mass less than 300 daltons, less than 4 hydrogen bond donors and acceptors less than 4 rotatable bonds were filtered and the resulting molecule set of 27,200 compounds was subjected to molecular docking calculations.

Structure-based vHTS on FAK H58: In all computational steps, Linux shell scripts and php scripts written by us or part of DockingServer allowing mass data handling were applied. The protonation state of ligands at neutral pH was calculated using JChem. Gasteiger partial charges were applied on the ligand and protein atoms. Rotatable bonds during docking calculations were identified with MGL Tools. Autodock Vina software was applied for molecular docking calculations. All rotatable bonds of the ligands were treated flexible. Docking interactions were calculated in a simulation box with a dimension of 20×20×20 Å centered around H58. Exhaustiveness options was set to 8. Docking simulations were run on a 16-core Linux workstation. First rank docking energies and geometries were considered in further analysis.

The results were analyzed from both of lowest docking energy and interaction pattern analysis. Interaction pattern analysis included calculation of hydrogen bonds and apolar interactions between the protein and the docked ligands. As the goal of the study was to identify ligands that bind to H58, the presence of this interaction was used as the main filter in results evaluation. 1502 molecules were calculated to form hydrogen bonding interactions with H58. The molecules were then further ranked by their docking energy.

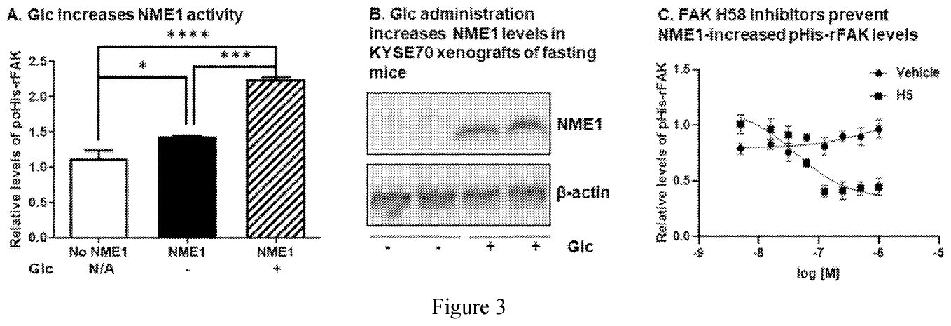

FAK^{His58} lead (H5) attenuates NME1-catalyzed FAK^{pHis}. Glc increases NME1 activity in cells and expression in mice xenografts, as shown in FIGS. 3A-3B, which contributes to Glc-induced proliferation. Consistent with a role in promoting Glc-induced pHis levels in ESCC, H5 prevented Glc-mediated pHis-protein induction, as shown in FIG. 3C, strongly suggesting that FAK^{His58} leads such as H5 can act as novel NME1 histidine kinase inhibitors that prevent FAK^{pHis58} in Glc-induced ESCC growth.

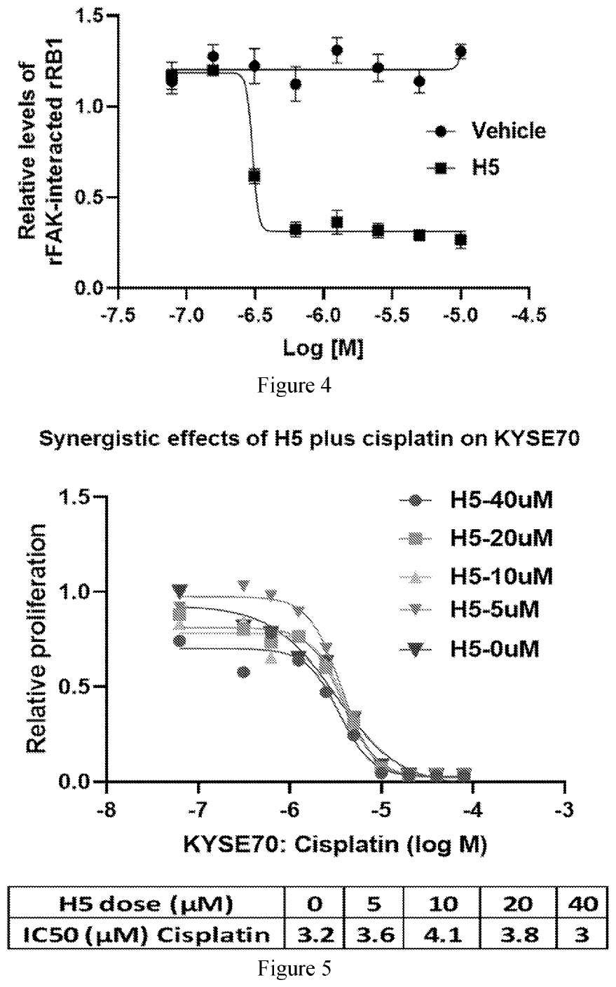

H5 prevents Glc-induced FAK-RB1 interaction. Previously, it was identified that RB1 associates with FAK, but only after Glc treatment. The data shown in FIG. 4, therefore, provides further support for the present technology inasmuch as H5 specifically selects against FAK^{His58} thereby interrupting the FAK-RB1 interaction.

Lead compound and more potent analogues prevent ESCC proliferation. MTT-based assessments of FAK^{His58} inhibitor-attenuated ESCC proliferation include: FAK^{His58} inhibitor [H5: 5,6-diphenyl-1,2,4-triazin-3(2H)-one], which decreased proliferation of three ESCC cell lines (KYSE70, TE9, and TE10). The IC50 values are shown in Table 2.

TABLE 2
IC⁵⁰ of H5 on ESCC cells were incubated in medium containing
5% FBS and varied concentrations (1 nM to 1 μM) of H5 for 48 hr
and subjected to WST-1 assay. IC⁵⁰ values were calculated.

| IC⁵⁰ (μM) | TE9 | TE10 | KYSE70 |
|---|---|---|---|
| H5 | 0.38 | 0.088 | 5.7 |

H5 derivatives have high potency H5A: 6,7-diphenyl-2H-[1,2,4]triazolo[4,3-b][1,2,4]triazine-3-thione, and H5B: ethyl [(5,6-diphenyl-1,2,4-triazin-3-yl)thio]acetate were assessed for their potency as shown in Table 3. These H5 derivatives prevented ESCC (TE10) cell proliferation with an IC50 value as low as 1.5 nM for H5A. These results strongly support that the novel FAK^{pHis58} inhibitors with high potency and efficacy can be developed and used to prevent Glc-promoted ESCC growth following optimization, selection, and IND-enabling studies.

TABLE 3
IC⁵⁰ of representative H5 analogs. TE10 cells were incubated in medium containing 5% FBS and varied
concentrations of H5 analogs for 48 hr and subjected to WST-1 assay. IC⁵⁰ values were calculated.

| | 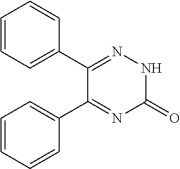 | 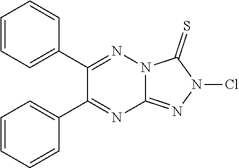 | 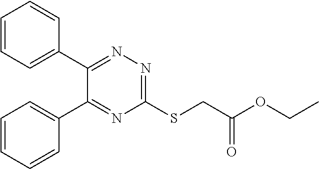 |
|---|---|---|---|
| IC⁵⁰ (μM) | H5 | H5A | H5B |
| TE10 | 0.088 | 0.0015 | 0.065 |

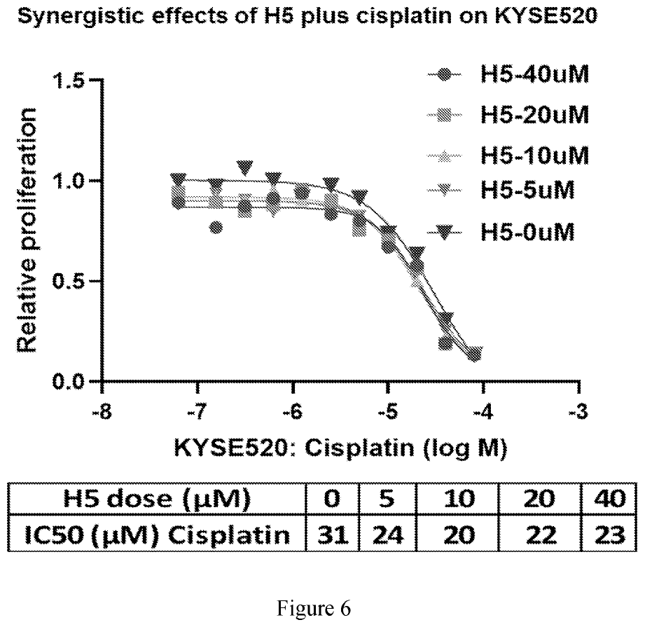

Synergistic effects of the chemotherapy drug combined with FAK H58 inhibitor. Chemotherapies are commonly used treatments for patients with ESCC. It was sought to determine whether the inhibition of glucose-induced G1-to-S phase transition of the cell cycle coupled with the interruption of DNA synthesis by chemotherapeutic agents is synergistic. ESCC cells (KYSE70 and KYSE520) were cultured in the medium containing reduced FBS (5%), 0-80 μM of cisplatin and 5 fixed doses of H5 (0, 5, 10, 20, or 40 μM) for 72 hr. Cell proliferation was assessed using WST1, a MTT-like reagent. The IC50 values of cisplatin were comparable in KYSE70, when cisplatin was combined with varied doses of FAK H58 inhibitor H5 (FIG. 5). Small molecule H5 decreased IC50 of cisplatin from 31 to 23 μM in KYSE520 cells (FIG. 6). This suggests that blocking glucose-induced cell cycle progression enhanced the effects of cisplatin on KYSE520 cell proliferation (FIG. 6).

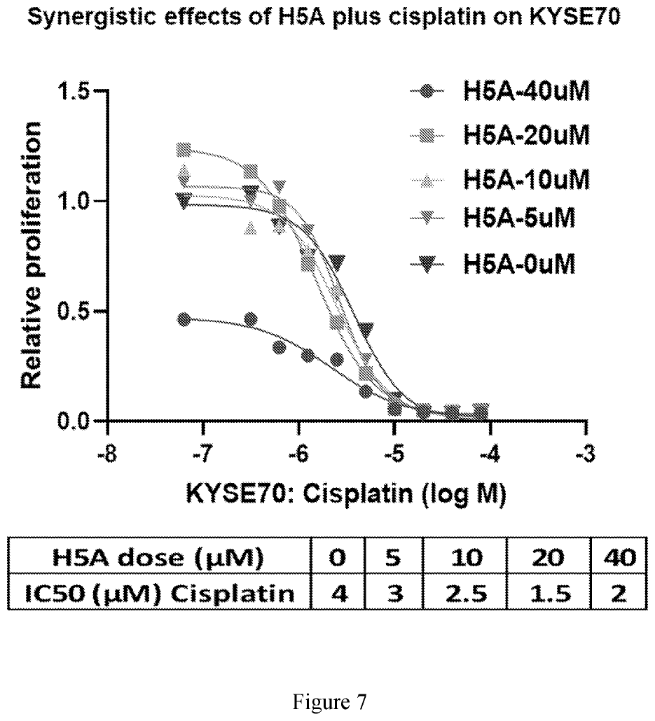

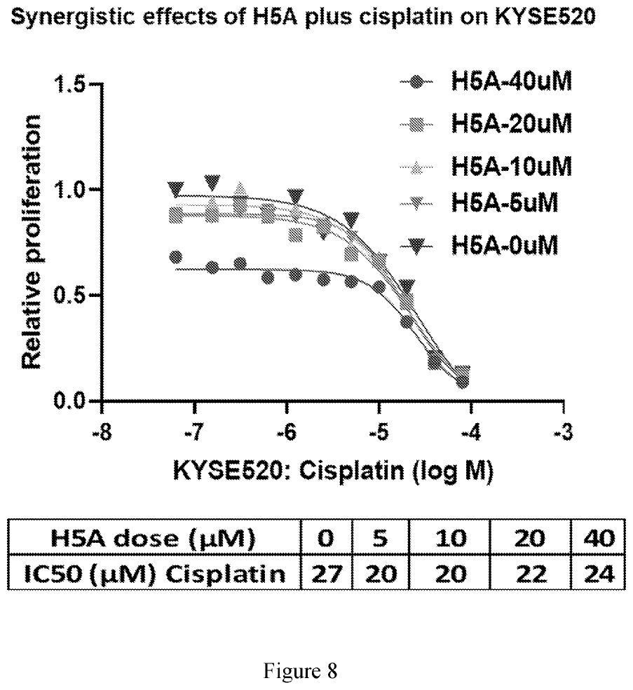

H5 Å was more potent than its parent compound H5 (FIGS. 2 and 3). To assess the synergistic effects of cisplatin coupled with the FAK H58 inhibitor derivative (H5A), ESCC cells (KYSE70 and KYSE520) were treated with 0-80 μM of cisplatin and 5 fixed doses of H5 Å for 72 hr. The H5 derivative H5 Å enhanced the inhibitory effects of cisplatin in KYSE70 from 4 to 2 μM (FIG. 7) and in KYSE520 from 27 to 24 μM (FIG. 8), respectively. This suggests that the combination of cisplatin and H5 Å was synergistic. These results demonstrate that combination therapies of current chemo-drugs and the novel FAK H58 inhibitor can be developed and used to prevent ESCC growth.

Validation of the vHTS hits: The accuracy of docking calculation was confirmed by redocking of the hit structures to FAK H58, clustering the ligands with similar interactions, ranking the hits based on their calculated affinities.

Reproducible test: BrdU coupling ELISA, pHis-FAK assays and IP/IB were performed to confirm the FAK H58 hit inhibition of ESCC proliferation, phosphorylation of FAK on H58 and FAK-RB1 interaction (FIGS. 2-4).

Dose response curve: Top ranked hits based on their H58 interaction, inhibition of proliferation, blocking NME1 phosphorylation of H58 and interruption of FAK-RB1 binding were assessed for their dose response effect on Glc-promoted DNA synthesis, FAK H58 phosphorylation and FAK-RB1 interaction, respectively (FIGS. 2-4).

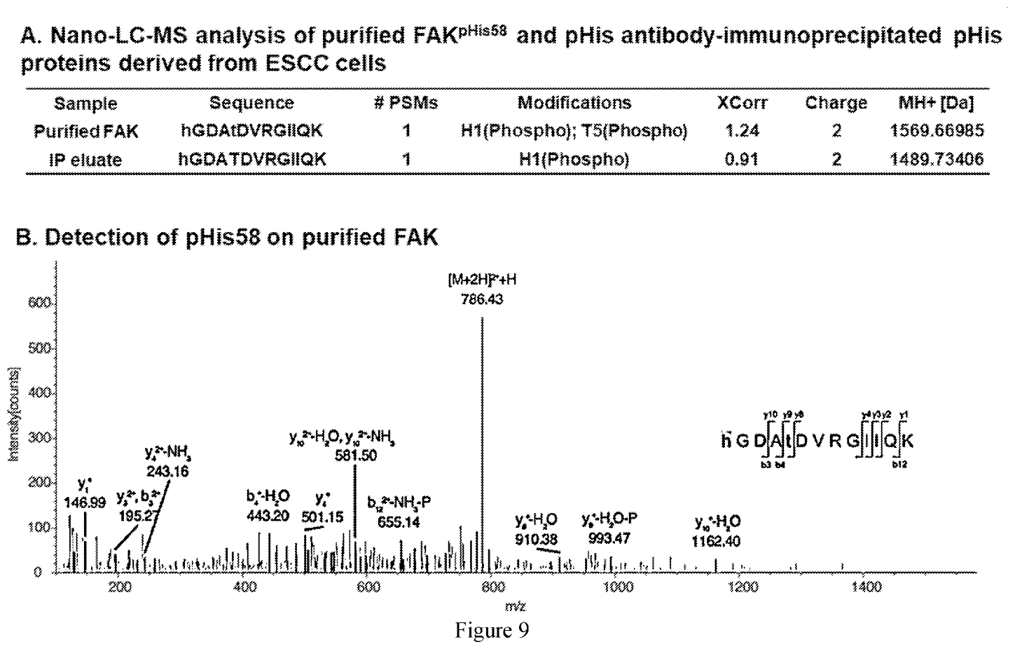

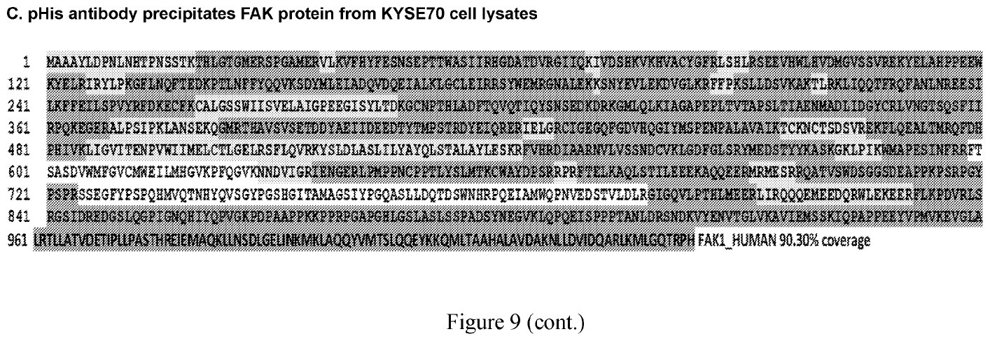

poHis58 FAK: Mass spectroscopy was performed to confirm H58 phosphorylation of recombinant FAK protein and FAK isolated from ESCC cells with or without Glc stimulation. These data are shown in FIG. 9.

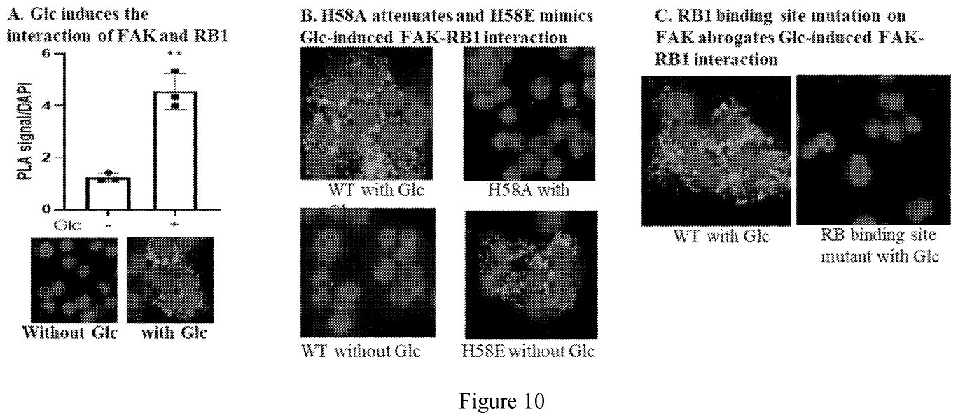

FAK-RB1 interaction: Proximity ligation assay (PLA) was carried out to verify FAK-RB1 interaction in ESCC cells. These data are shown FIG. 10.

The present disclosure provides i) a tumor system describing how Glc can act as a sole mitogenic driver, ii) active leads that inhibit Glc-stimulated FAK^{poHis58} by inhibition of NME1-catalyzed histidine phosphorylation, and iii) small molecules that interrupt Glc-induced FAK-RB1^{pos780} interaction and proliferation. These data incorporate a heretofore undescribed role of FAK H58 inhibitors for targeting a novel histidine phosphorylation pathway in controlling ESCC proliferation, yet not induced by a GF but by Glc as a sole mitogen.

Small molecule inhibition of glucose-promoted ESCC growth: These studies indicate a direct inhibition of onco-proliferative function of Glc in ESCC. These tumors have diverse genetic abnormalities but may share a common metabolic weakness, namely that their GF-independence is Glc-dependent. This new approach can help resolve fundamental dilemmas: how to attack the driving force of uncontrolled ESCC growth; and how to overcome the obstacle that clinical therapies targeting RTKs have had limited or no effect. The Glc levels required to induce ESCC proliferation is roughly 160-fold lower than those found in normal blood, suggesting that Glc functions in ESCC as a GF-like mitogen. In addition, Glc is an essential nutrient for normal cells to live and grow. Current attempts to block Glc uptake have had limited success due to several side effects. This approach of targeting Glc-induced growth signaling but not Glc uptake/metabolism will prevent ESCC growth with relatively low toxicity.

Targeting poHis signaling: Glc→FAK^{poHis} signaling fills the knowledge gap between excessive Glc metabolism via glycolysis (to increase PEP levels and trigger alternative phosphohistidine signaling) and ESCC growth. Current FAK-targeted or other kinase inhibitors are typically ATP-competitive compounds or inhibitors of scaffolding activity with signaling partners, and they are usually assessed for inhibition of GF-induced signaling and/or proliferation. However, these studies indicate that Glc-induced FAK^{poHis58}_dependent ESCC proliferation does not require FAK-Y397 phosphorylation, a typical GF/RTK-induced mitogenesis event, suggesting that these efforts to target NME1 and FAK^{poHis58}-RB1 interaction will inhibit ESCC growth, which have particularly evolved GF-independent pathways.

Although the present disclosure has been described with respect to one or more particular examples, it will be understood that other examples of the present disclosure may be made without departing from the scope of the present disclosure.

1. A compound having the structure: 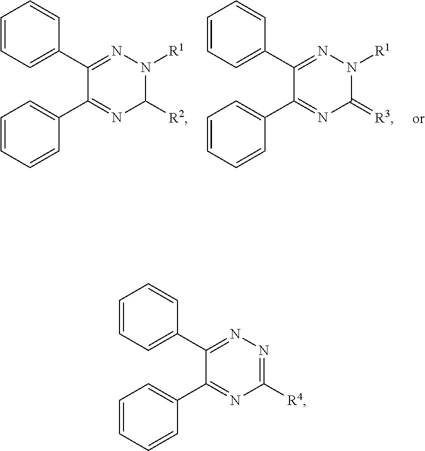
wherein
R¹ is chosen from H, substituted or unsubstituted aliphatic groups, and substituted or unsubstituted aryl groups;
R³ is a double bonded S or O;
R⁴ is a substituted or unsubstituted alkyl group or substituted or unsubstituted heteroalkyl group;
or
R¹ and R² are combined to form a substituted or unsubstituted carbocyclic ring, a substituted or unsubstituted heterocyclic ring, a substituted or unsubstituted aryl ring, or a substituted or unsubstituted heteroaryl ring;
or
R¹ and R³ are combined to form a substituted or unsubstituted carbocyclic ring, a substituted or unsubstituted heterocyclic ring, a substituted or unsubstituted aryl ring, or a substituted or unsubstituted heteroaryl ring.

2. The compound of claim 1, wherein R¹ and R² or R¹ and R³ combine to form a substituted or unsubstituted 5-membered carbocyclic, heterocyclic, or heteroaryl ring; substituted or unsubstituted 6-membered carbocyclic, heterocyclic, aryl, or heteroaryl ring, or a substituted or unsubstituted 7-membered carbocyclic or heterocyclic ring.

3. The compound of claim 1, having the following structure:

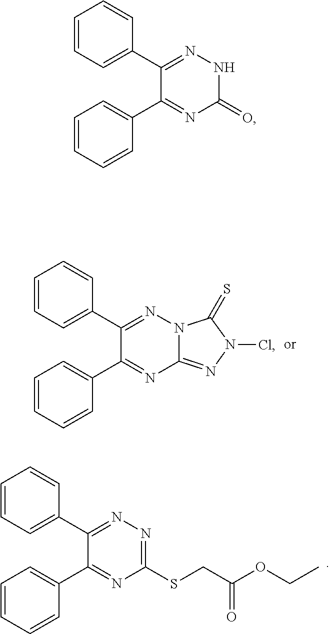 [SMILES: O=c1nc(-c2ccccc2)c(-c2ccccc2)n[nH]1 ; S=c1n(Cl)nc2nc(-c3ccccc3)c(-c3ccccc3)nn12 ; CCOC(=O)CSc1nnc(-c2ccccc2)c(-c2ccccc2)n1]

4. A composition comprising a compound of claim 1 and a pharmaceutically acceptable carrier.

5. The composition of claim 4, wherein the compound has the following structure:

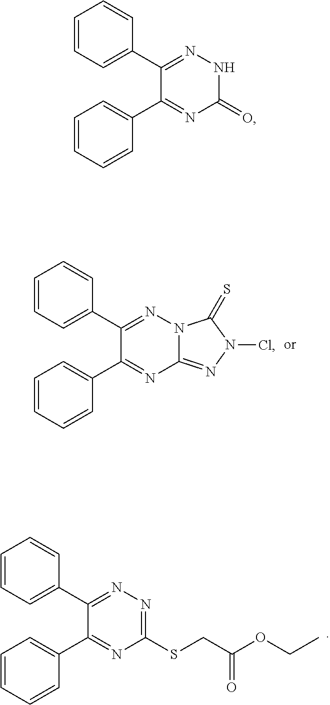 [SMILES: O=c1nc(-c2ccccc2)c(-c2ccccc2)n[nH]1 ; S=c1n(Cl)nc2nc(-c3ccccc3)c(-c3ccccc3)nn12 ; CCOC(=O)CSc1nnc(-c2ccccc2)c(-c2ccccc2)n1]

6. A method for treating an individual having cancer or suspected of having cancer, comprising administering to the individual a therapeutically effective amount compound of claim 1.

7. The method of claim 6, wherein the cancer is a carcinoma of the lung, mucous membranes, or urinary tract, wherein the carcinoma of lung, mucous membranes, or urinary tract have cancerous squamous cells with Glc-induced growth signaling.

8. The method of claim 6, wherein the cancer is drug-resistant esophageal squamous cell carcinoma or esophageal squamous cell carcinoma.

9. The method of claim 6, wherein the compound has the following structure:

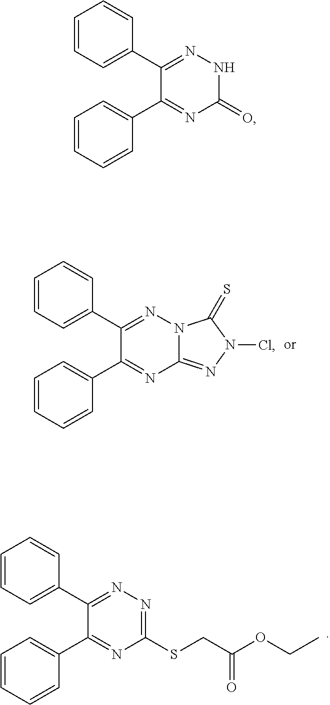 [SMILES: O=c1nc(-c2ccccc2)c(-c2ccccc2)n[nH]1 ; S=c1n(Cl)nc2nc(-c3ccccc3)c(-c3ccccc3)nn12 ; CCOC(=O)CSc1nnc(-c2ccccc2)c(-c2ccccc2)n1]

10. The method of claim 6, further comprising surgery, radiation therapy, chemotherapy, photodynamic therapy, and/or immunotherapy.

11. A compound having the following structure:

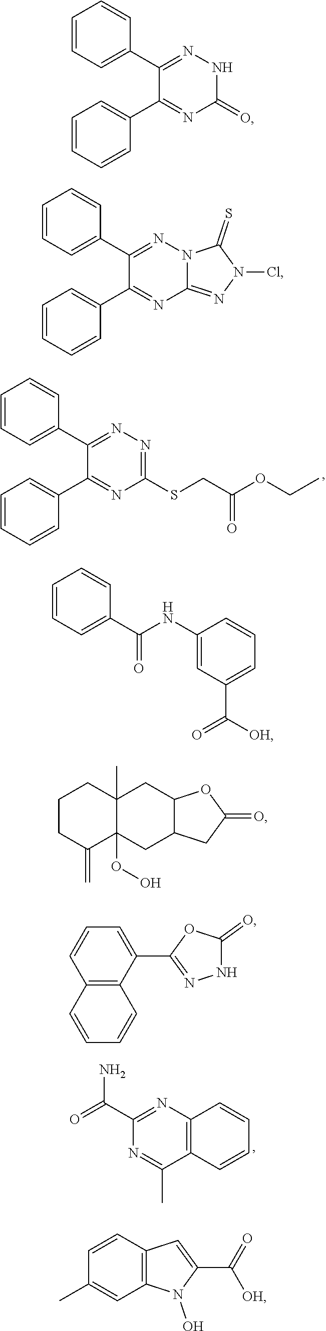

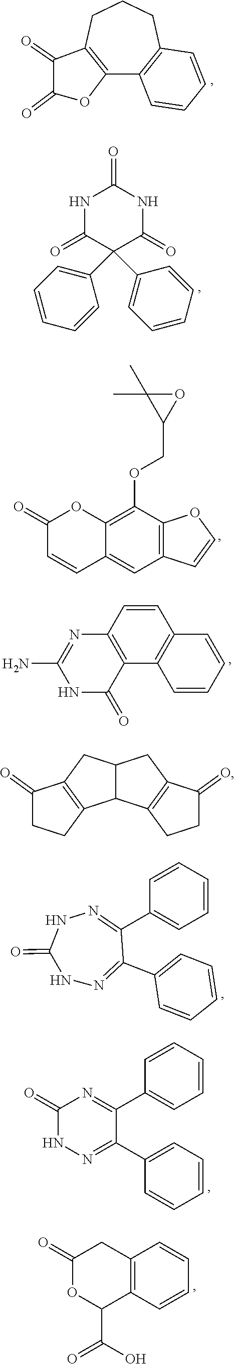

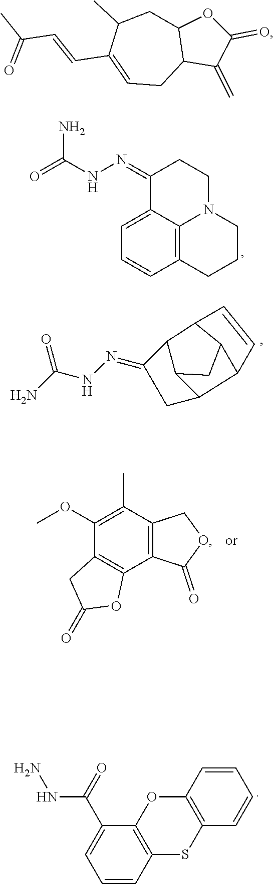

12. A composition comprising a compound of claim 11 and a pharmaceutically acceptable carrier.

13. A method for treating an individual having cancer or suspected of having cancer, comprising administering to the individual a therapeutically effective amount compound of claim 11.

14. The method of claim 13, wherein the cancer is a carcinoma of the lung, mucous membranes, or urinary tract, wherein the carcinoma of lung, mucous membranes, or urinary tract have cancerous squamous cells with Glc-induced growth signaling.

15. The method of claim 13, wherein the cancer is drug-resistant esophageal squamous cell carcinoma or esophageal squamous cell carcinoma.

16. The method of claim 13, further comprising surgery, radiation therapy, chemotherapy, photodynamic therapy, and/or immunotherapy.

17. A method for inhibiting glucose-induced growth signaling, comprising administering a therapeutically effective amount of a compound of claim 1, wherein glucose uptake or metabolism is not inhibited.

18. A method for inhibiting glucose-induced growth signaling, comprising administering a therapeutically effective amount of a compound of claim 11, wherein glucose uptake or metabolism is not inhibited.


SEQUENCE LISTING (2 sequences)

SEQ ID NO: 1 - protein, 13 aa, Artificial Sequence
  FAK segment
HGDATDVRGIIQK

SEQ ID NO: 2 - protein, 1052 aa, Homo sapiens
MAAAYLDPNLNHTPNSSTKTHLGTGMERSPGAMERVLKVFHYFESNSEPTTWASIIRHGDATDVRGIIQKIVDSHKVKHVACYGFRLSHLRSEEVHWLHVDMGVSSVREKYELAHPPEEWKYELRIRYLPKGFLNQFTEDKPTLNFFYQQVKSDYMLEIADQVDQEIALKLGCLEIRRSYWEMRGNALEKKSNYEVLEKDVGLKRFFPKSLLDSVKAKTLRKLIQQTFRQFANLNREESILKFFEILSPVYRFDKECFKCALGSSWIISVELAIGPEEGISYLTDKGCNPTHLADFTQVQTIQYSNSEDKDRKGMLQLKIAGAPEPLTVTAPSLTIAENMADLIDGYCRLVNGTSQSFIIRPQKEGERALPSIPKLANSEKQGMRTHAVSVSETDDYAEIIDEEDTYTMPSTRDYEIQRERIELGRCIGEGQFGDVHQGIYMSPENPALAVAIKTCKNCTSDSVREKFLQEALTMRQFDHPHIVKLIGVITENPVWIIMELCTLGELRSFLQVRKYSLDLASLILYAYQLSTALAYLESKRFVHRDIAARNVLVSSNDCVKLGDFGLSRYMEDSTYYKASKGKLPIKWMAPESINFRRFTSASDVWMFGVCMWEILMHGVKPFQGVKNNDVIGRIENGERLPMPPNCPPTLYSLMTKCWAYDPSRRPRFTELKAQLSTILEEEKAQQEERMRMESRRQATVSWDSGGSDEAPPKPSRPGYPSPRSSEGFYPSPQHMVQTNHYQVSGYPGSHGITAMAGSIYPGQASLLDQTDSWNHRPQEIAMWQPNVEDSTVLDLRGIGQVLPTHLMEERLIRQQQEMEEDQRWLEKEERFLKPDVRLSRGSIDREDGSLQGPIGNQHIYQPVGKPDPAAPPKKPPRPGAPGHLGSLASLSSPADSYNEGVKLQPQEISPPPTANLDRSNDKVYENVTGLVKAVIEMSSKIQPAPPEEYVPMVKEVGLALRTLLATVDETIPLLPASTHREIEMAQKLLNSDLGELINKMKLAQQYVMTSLQQEYKKQMLTAAHALAVDAKNLLDVIDQARLKMLGQTRPH